(19) United States
(12) Patent Application Publication
(10) Pub. No.: US 2025/0114534 A1
(43) Pub. Date: Apr. 10, 2025

(54) PEN INJECTION DEVICE DOSE INDICATION APPARATUS AND METHOD

(71) Applicant: Becton, Dickinson and Company, Franklin Lakes, NJ (US)

(72) Inventors: Roger Groskopf, Saddle Brook, NJ (US); Heesang Lee, Montvale, NJ (US); Keith Pierce, Maplewood, NJ (US)

(73) Assignee: Becton, Dickinson and Company, Franklin Lakes, NJ (US)

(21) Appl. No.: 18/728,757

(22) PCT Filed: Jan. 12, 2023
(86) PCT No.: PCT/US2023/010718

§ 371 Date: Jul. 12, 2024

Related U.S. Application Data
(60) Provisional application No. 63/299,204, filed on Jan. 13, 2022.

(51) Int. Cl. A61M 5/315

(52) U.S. Cl. CPC A61M 5/31528

(57) ABSTRACT

A medication injection device and method of setting a desired dose of medication are provided, where the device includes a housing having an opening or a window therein, and a dose set knob is disposed at least partially within the housing. The dose set knob is movable with respect to the housing to set a desired dose of medication and has represented thereon one or more dose value indicators. The opening or window in the housing and the dose value indicators on the dose set knob are configured such that, when moving the dose set knob with respect to the housing to set or select the desired dose, the dose value representative of the selected dose is legible when viewed through the opening or window, but otherwise is not clearly legible.

### BACKGROUND

### 1. Field

Apparatuses and methods consistent with example embodiments relate to a multiple use pen-type injection device with dose setting capability, as well as dial back of a set dose, and/or last dose control.

### 2. Description of the Related Art

Various medication injection pen devices are known. Such devices may include features for enabling a user to set a dose and/or to correct a dose that has been set too large, which may be referred to as “dial back.” Another feature that may be provided is an ability to control a last dose of a medication cartridge such that a user cannot set a dose greater than a remaining amount of medication in the cartridge. This feature may be referred to as last dose control or last dose management. Various pen devices incorporating one or more of these, and other features, are described in prior patents including, without limitation U.S. Pat. Nos. 9,295,782; 9,421,334; 9,757,525; 10,357,614; and 10,661,021; and U.S. Published Patent Application Nos. 2020-0282147 and 2021-0299359, the entire disclosures of all of which are incorporated herein by reference.

Referring to FIG. 1, a pen-type injection device which has a dose dial set, and optionally a dial back feature, may include a pen upper body or housing 1, which houses a plurality of dose setting and injection components including a dose set knob 2. The pen upper body 1 can be connected to a cartridge housing 14, which houses a medication cartridge. The injection pen may also include a cover to cover a needle attached to the cartridge when the injection pen is not in use.

Referring to FIGS. 2A and 2B A dose set knob 2 can includes a knob-like portion that is rotated by a user to set a desired dose, and another portion axially movable within housing 1 having one or more numerals 3000 corresponding to a number of dosage units, that may be visible through a dose set window 13 provided in the housing 1. The pen upper body 1 may also include an arrow or other indicator 53 to point to a numeral 3000 in window 13. A user would typically rotate the dose set knob 2 until the numeral 3000 corresponding to a desired dose is visible in the window 13 and then inject the set dosage amount by pressing button 3.

Having both dose setting and dial back features for setting a desired dose is favored by users of such pen devices. However, users of current pen type devices typically configured as shown in FIGS. 1 and 2 may possibly misread a set dose value, for example by observing corresponding numeral 3000 from an outside of the window 13, such as when the does set knob 2 is in a position as shown in FIG. 2A. This possible misreading may be prevented according to one or more example aspects as described below.

### SUMMARY

Example embodiments may address at least the above problems and/or disadvantages and other disadvantages not described above. Also, example embodiments are not required to overcome the disadvantages described above, and may not overcome any of the problems described above.

One or more example embodiments may provide an injection device comprising a dose set window including a cover or a filter which, in combination with a certain representation of a dose value, shows an indicator representing a desired set dose when seen by a user through the dose set window. The representation of the dose value appears unclear or as not a meaningful indicator when viewed on its own, or outside the dose set window.

According to one or more implementations of example embodiments, an injection device may comprise a dose set knob movable by a user to set the desired dose. The dose set knob includes one or more representations of the dose value thereon, such that the movement of the dose set knob causes movement of the representations of the dose value with respect to the dose set window. When the desired dose is set by movement of the dose set knob, an indicator representing the desired set dose is seen by a user through the dose set window.

According to an aspect of an example embodiment, a medication injection method comprises: axially moving a dose set knob of a medication injection pen with respect to a housing of the medication injection pen, thereby displaying a selected dose of a plurality of dose values represented on the dose set knob, through a window in the housing; and injecting the selected dose; wherein the window comprises an opening in the housing and a cover over the opening, and the displaying the selected dose comprises displaying the selected through the cover.

According to an aspect of another example embodiment, a medication injection pen comprises: a housing having a window formed therein; a dose set knob, disposed at least partially within the housing, that moves axially with respect to the housing, the dose set knob comprising a plurality of dose values represented thereon; wherein the window comprises an opening in the housing and a cover over the opening, and a combination of the cover and one of the plurality of dose values represented on the dose set knob legibly displays a selected dose.

The dose set knob may be rotatable with respect to the housing.

The medication injection pen may further comprise: a driver engaged with the dose set knob and rotatably fixed with respect to the housing during dose setting; wherein the driver moves axially with the dose set knob during dose setting, and the driver rotates with the dose set knob during an injection.

The dose set knob may rotates to cause the driver to rotate during an injection.

The medication injection pen may further comprise a piston rod rotatably fixed to the housing, the piston rod selectively connected to the dose set knob.

The cover may comprises one of an opaque cover having seven transparent portions therein; a polarized filter; a magnifying filter; and a color filter.

The cover may comprise a convex lens.

The cover may comprise an opaque cover having seven openings formed therethrough.

The cover may comprise a transparent cover and an opaque mask having seven openings formed therethrough.

### BRIEF DESCRIPTION OF THE DRAWINGS

The above and/or other aspects will become apparent and more readily appreciated from the following description of example embodiments, taken in conjunction with the accompanying drawings in which:

FIG. 1 is an example injection pen with a dose dial window;

FIGS. 2A and 2B are detailed view of dose dial portion of injection pen of FIG. 1;

FIGS. 3A-3D show perspective views of an example embodiment of an injection pen including dose indication features according to example aspects of the disclosure;

FIG. 3E is a detailed view of example implementation shown in FIG. 3A-3D;

FIGS. 4A-4D show perspective views of still another example embodiment of an injection pen including dose indication features according to example aspects of the disclosure;

FIG. 4E is a detailed view of example implementation shown in FIG. 4A-4D

FIGS. 5A-5C show perspective views of another example embodiment of an injection pen including dose indication features according to further example aspects of the disclosure;

FIGS. 6A-6C show perspective views of yet another example embodiment of an injection pen including dose indication features according to still further example aspects of the disclosure;

FIG. 6D is a conceptual view of an example implementation for an example embodiment illustrated in FIG. 6A-6C;

FIG. 7 is a exploded assembly view of an injection pen according to an example embodiment;

FIG. 8 is an elevation view in cross-section of the injection pen of FIG. 7;

FIG. 9 is a perspective view of a dose set knob of the injection pen of FIG. 7;

FIG. 10 is an elevation view in cross-section of the dose set knob of FIG. 9;

FIG. 11 is a perspective view of a setback member of the injection pen of FIG. 7;

FIG. 12 is an elevation view in cross-section of the setback member of FIG. 11;

FIG. 13 is a distal perspective view of the setback member of FIG. 11;

FIG. 14 is a perspective view of a lead screw of the injection pen of FIG. 7;

FIG. 15 is a perspective view of a tower core of the injection pen of FIG. 7;

FIG. 16 is a perspective view of a brake tower of the injection pen of FIG. 7;

FIG. 17 is an elevation view in cross-section of the brake tower of FIG. 7;

FIG. 18 is a perspective view of a piston rod of the injection pen of FIG. 7;

FIG. 19 is an elevation view in cross-section of the piston rod of FIG. 18;

FIG. 20 is a perspective view of a pen upper body of the injection pen of FIG. 7;

FIG. 21 is an elevation view in cross-section of the pen upper body of FIG. 20;

FIG. 22 is a perspective view of a clicker body of the injection pen of FIG. 7;

FIG. 23 is a bottom plan view of the clicker body of FIG. 22;

FIG. 24 is an elevation view of the clicker body of FIG. 22;

FIG. 25 is a top plan view of the clicker body of FIG. 22;

FIG. 26 is a proximal perspective view of the brake tower of the injection pen of FIG. 7;

FIG. 27 is a perspective view of the lead screw and tower core prior to engagement with the brake tower of the injection pen of FIG. 7

FIG. 28 is a perspective view of the lead screw connected to the tower core prior to being connected to the brake tower of FIG. 27;

FIG. 29 is an elevation view of the lead screw and tower core connected to the brake tower of FIG. 27;

FIG. 30 is an elevation view of the engagement between the piston rod and tower core of the injection pen of FIG. 7;

FIG. 31 is an elevation view in cross-section of a clicker body disposed between a dose set knob and a setback member of an injection pen in accordance with an eighth example embodiment;

FIG. 32 is a perspective view of the clicker body of FIG. 31;

FIG. 33 is partial perspective view of the setback member of the injection pen of FIG. 31;

FIG. 34 is a partial perspective view of the dose set knob of the injection pen of FIG. 31; and

FIG. 35 is an elevation view in cross-section of the injection pen of FIG. 31.

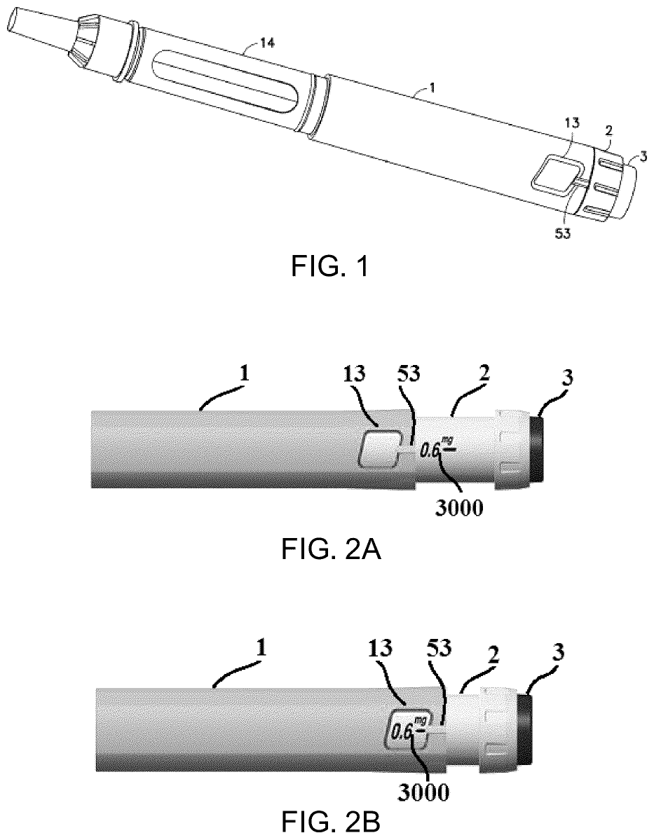

### DETAILED DESCRIPTION

Reference will now be made in detail to example embodiments which are illustrated in the accompanying drawings, wherein like reference numerals refer to like elements throughout. In this regard, the example embodiments may have different forms and may not be construed as being limited to the descriptions set forth herein.

It will be understood that the terms “include,” “including,” “comprise, and/or “comprising,” when used in this specification, specify the presence of stated features, integers, steps, operations, elements, and/or components, but do not preclude the presence or addition of one or more other features, integers, steps, operations, elements, components, and/or groups thereof.

It will be further understood that, although the terms “first,” “second,” “third,” etc., may be used herein to describe various elements, components, regions, layers and/or sections, these elements, components, regions, layers and/or sections may not be limited by these terms. These terms are only used to distinguish one element, component, region, layer or section from another element, component, region, layer or section.

As used herein, the term “and/or” includes any and all combinations of one or more of the associated listed items. Expressions such as “at least one of,” when preceding a list of elements, modify the entire list of elements and do not modify the individual elements of the list. In addition, the terms such as “unit,” “-er (-or),” and “module” described in the specification refer to an element for performing at least one function or operation, and may be implemented in hardware, software, or the combination of hardware and software.

Various terms are used to refer to particular system components. Different companies may refer to a component by different names-this document does not intend to distinguish between components that differ in name but not function.

Matters of these example embodiments that are obvious to those of ordinary skill in the technical field to which these example embodiments pertain may not be described here in detail.

According to example embodiments an injection device comprises a dose set window including a cover or filter which, in combination with specific indicators, such as printings, illustrates a number representing a dose when seen by a user through the cover or filter.

However, these indicators or printings appear unclear or as not a meaningful number when viewed on their own.

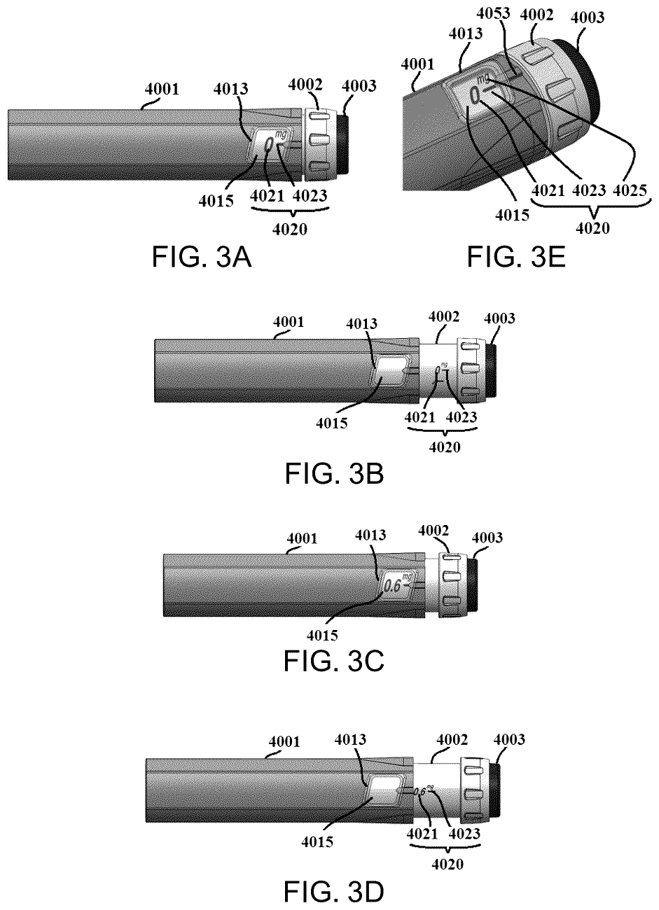

Referring to FIGS. 3A-3E, according to example embodiments, an injection device comprises a housing 4001 having a dose set window 4013 including a magnifying cover 4015, for example a convex lens, which magnifies an indicator or printed number 4020 positioned in the dose set window 4013 to a degree to make it legible to a user, as illustrated in the examples of FIGS, 3A, 3C, and 3E. One or more indicators or printed numbers 4020 can be disposed on a dose set knob 4002 of the injection device. When dose set knob 4002 is dialed out of, or positioned with respect to, housing 4001 such that the indicator or printed number 4020 is outside the window 4013, the indicator or printed number 4020 appears too small, less legible, or not legible due to being viewed without magnification provided by the magnifying cover 4015, as illustrated in the examples of FIGS. 3B and 3D. According to an example implementation, an indicator or printed number 4020 can comprise, for example and without limitation any combination of a numeric value 4021, a line or other pointer 4023, and/or a measurement unit indicator 4025, configured in any desired manner to be visible within window 4013 when a desired dose is set.

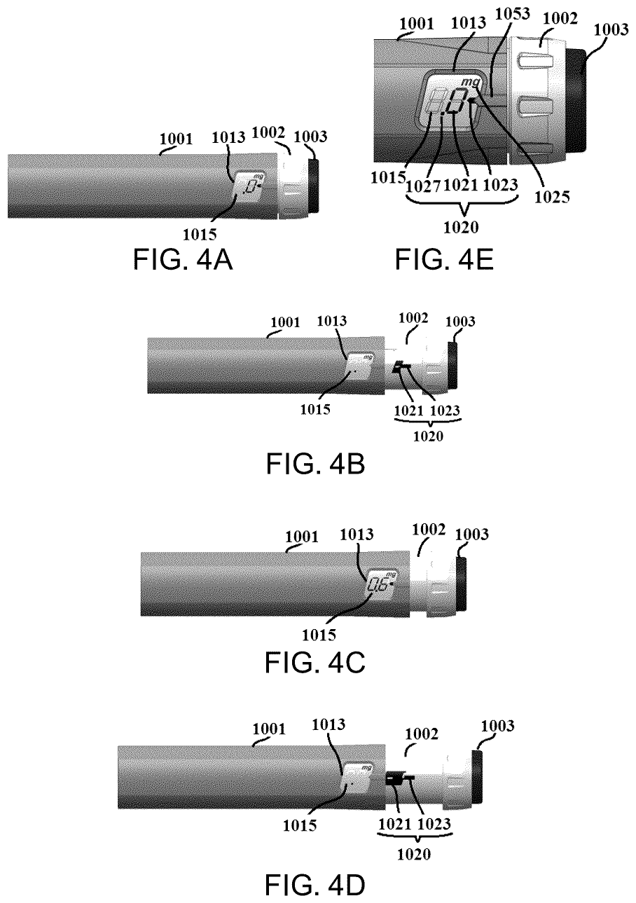

Referring to FIGS. 4A-4E, according to other example embodiments an injection device comprises a housing 1001 having a dose set window 1013 including a seven segment type window cover or filter 1015. In an exemplary implementation, seven segments are provided for each numerical value 1021 of the indicator 1020. One or more indicators 1020 can be disposed on a dose set knob 1002 of the injection device. This additional window cover or filter 1015 may be an opaque plastic including punctures indicating seven segments, or may be a transparent plastic masked by ink with transparent/unmasked portions for the seven segments, configured such that window cover or filter 1015 will only show seven segments from only one of the indicators 1020 when the indicator 1020 is positioned in the dose set window 1013, as illustrated in the examples of FIGS., 4A, 4C, and 4E. When dose set knob 1002 is dialed out of, or positioned with respect to, housing 1001 such that the indicator 1020 is outside the window 1013, the indicators, as seen outside the window 1013 or without filter 1015, do not form any meaningful numbers which could possibly be misread, as shown in FIGS. 4B and 4C. According to an example implementation, an indicator 1020 can comprise, for example and without limitation any combination of a numeric value 1021, a line or other pointer 1023, and/or, a decimal point 1027, configured in any desired manner to be visible within window 1013 when a desired dose is set. In an example implementation, a measurement unit indicator 1025 can be disposed on cover or filter 1015.

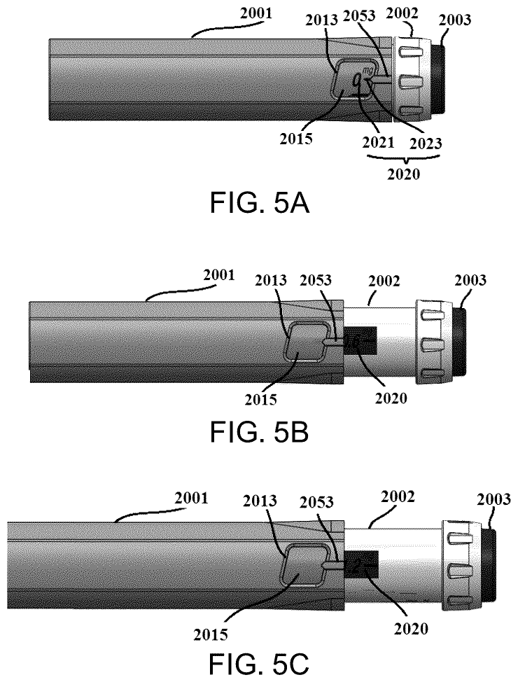

Referring to FIGS. 5A-5C, according to yet other example embodiments an injection device comprises a housing 2001 having a dose set window 2013 including a polarized filter 2015. One or more indicators 2020 can be disposed on a dose set knob 2002 of the injection device. Such a polarized filter 2015 is configured to show the dose unit to the user only through the polarized filter 2015. When dose set knob 2002 is dialed out of, or positioned with respect to, housing 2001 such that the indicator or printed number 2020 is outside the window 2013 or without filter 1015, the indicator or printed number 2020 will be less legible or will not show a meaningful number, as illustrated in the examples of FIGS. 5B and 5C. The number 2020 only appears clearly or meaningfully in window 2013 or through the filter 2015, as illustrated in the example of FIG. 5A. According to an example implementation, an indicator 2020 can comprise, for example and without limitation any combination of a numeric value 2021 and/or a line or other pointer 1023, configured in any desired manner to be visible within window 2013 when a desired dose is set.

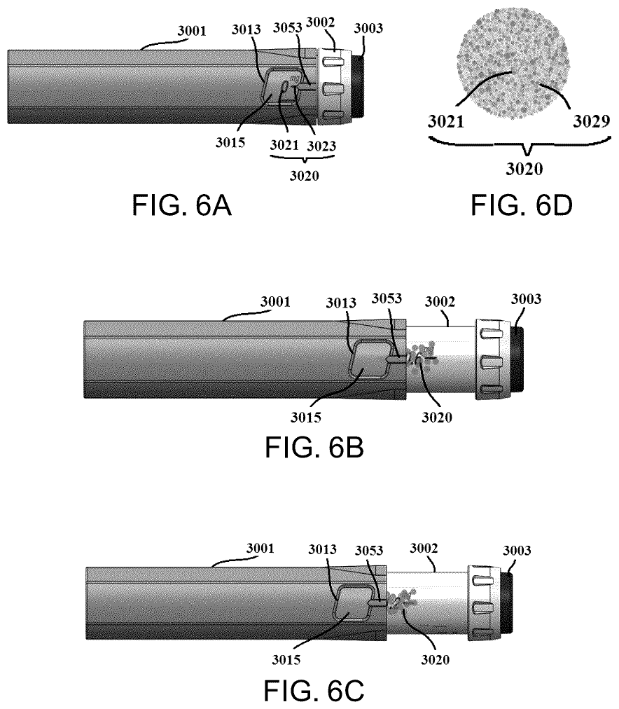

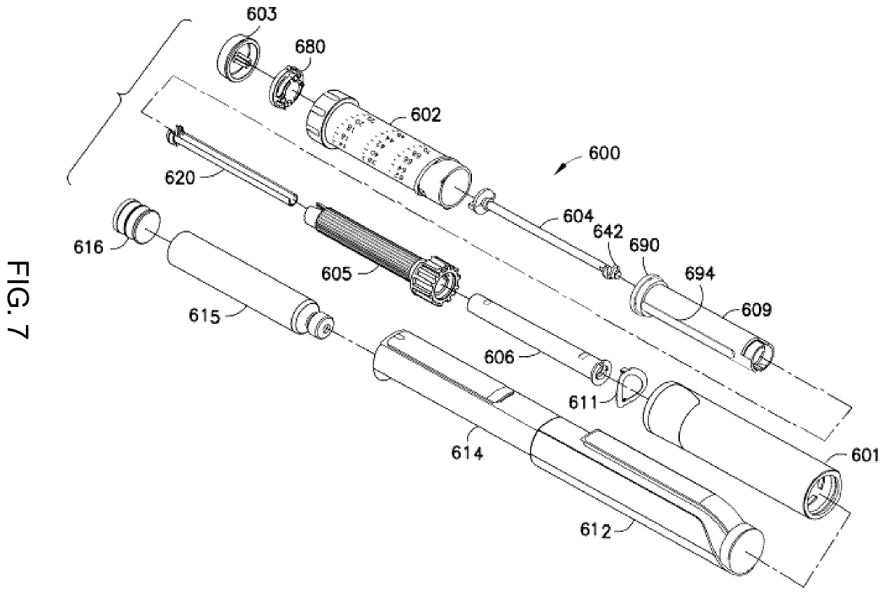

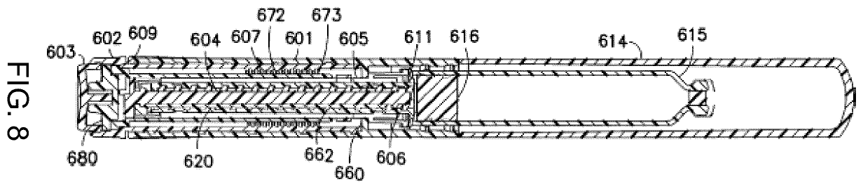

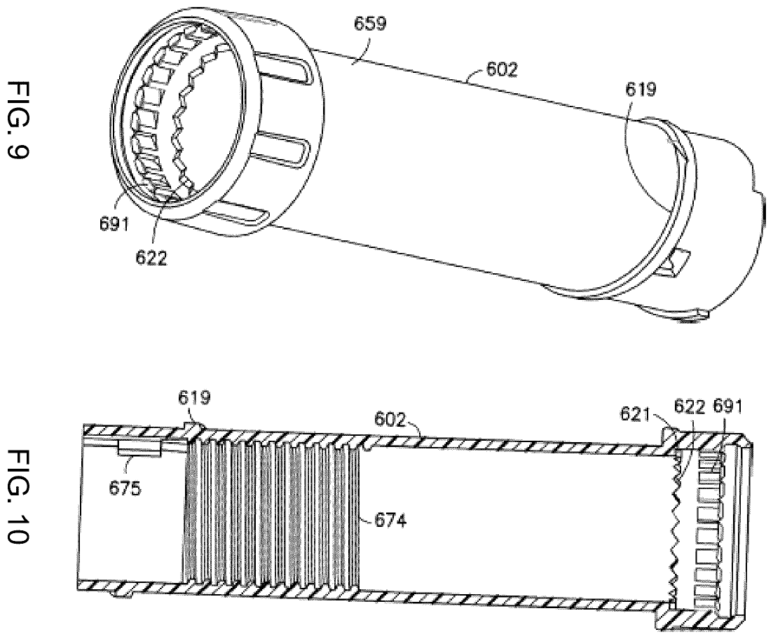

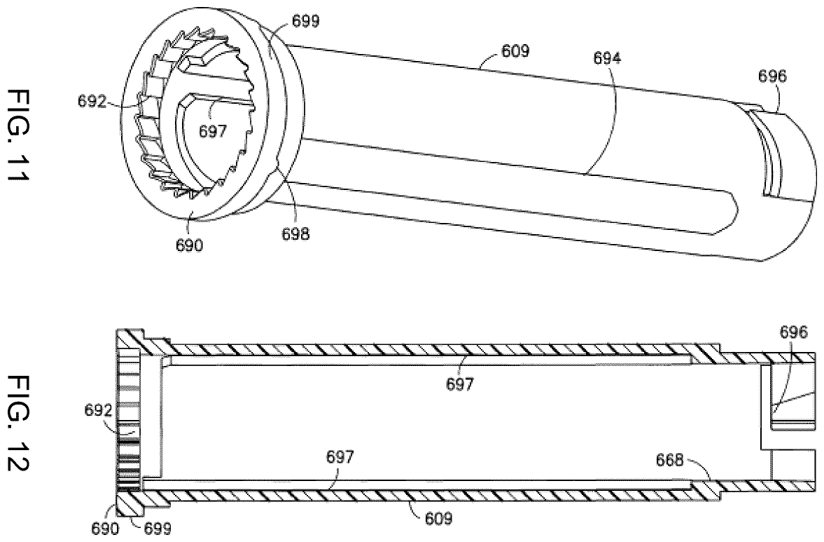

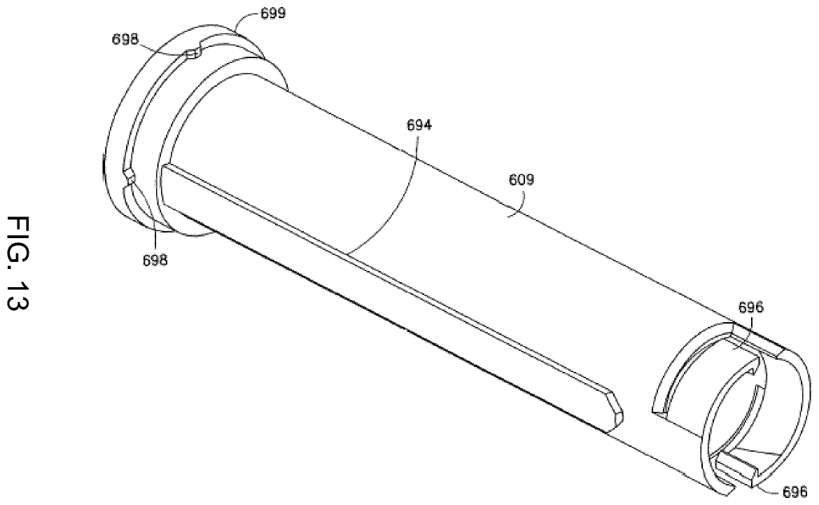

Referring to FIGS. 6A-6E, according to still further example embodiments an injection device comprises a housing 3001 having a dose set window 3013 including a color filter 3015. Such a color filter 3015 may be included over the window 3013, and an indicator 3020 may be printed to include a field of multiple colors, as illustrated in an example of FIG. 6D where indicator 3020 includes a number 3021 indicative of a dose within a colored field 3029. One or more indicators 3020 can be disposed on a dose set knob 3002 of the injection device. When dose set knob 3002 is dialed out of, or positioned with respect to, housing 3001 such that the indicator 3020 is outside the window 3013, the indicator 3020 will be less legible or will not show a meaningful number, as illustrated in the examples of FIGS. 6B and 6C. The number 3020 only appears clearly or meaningfully with the window 3013 or through the filter 3015, as shown in the example of FIG. 6A. According to an example implementation, an indicator 3020 can comprise, for example and without limitation any combination of a numeric value 3021 and/or a line or other pointer 3023, configured in any desired manner to be visible within window 3013 when a desired dose is set.

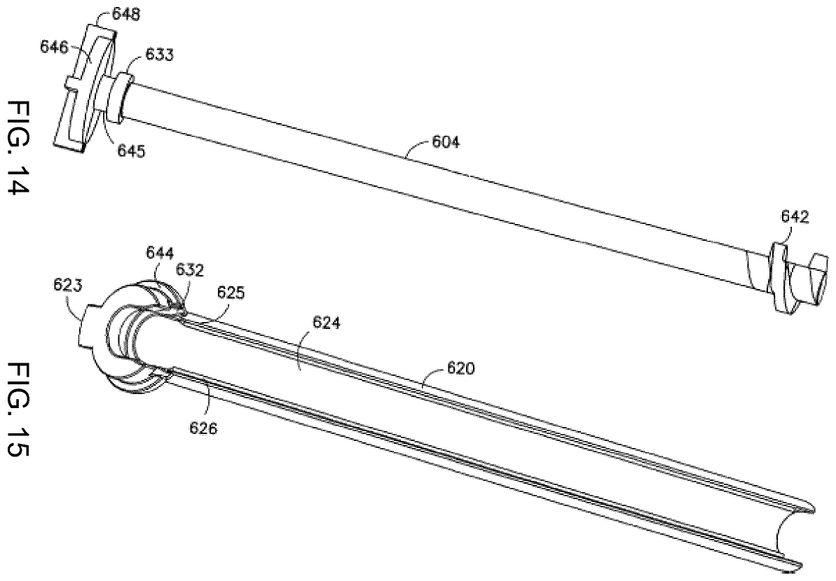

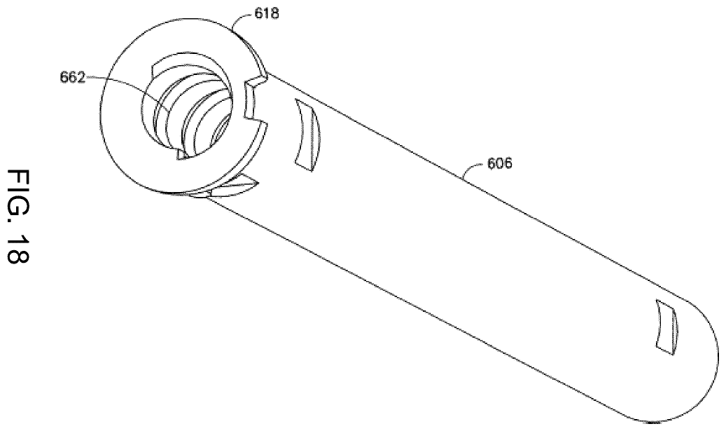

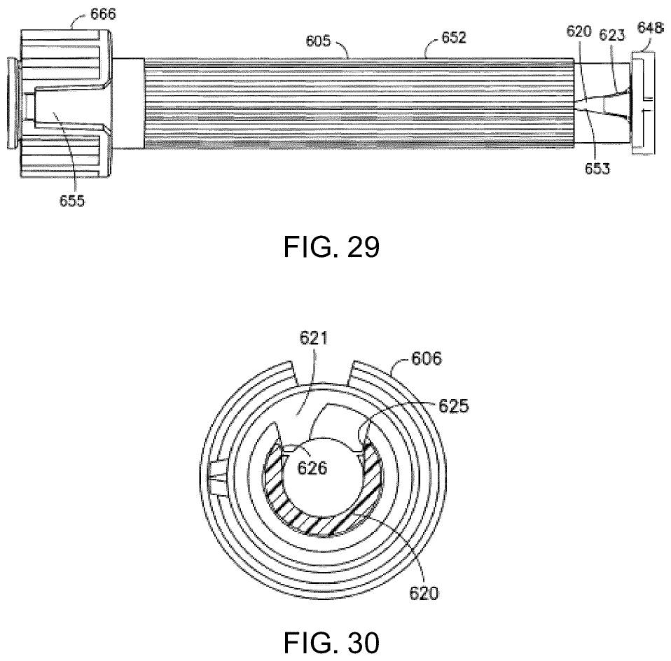

FIGS. 7-35 illustrate an example non-limiting embodiment of an injection pen 600 configured to implement any of the example embodiments disclosed and described above with reference to FIGS. 3A through 6E. As shown, the injection pen 600 includes a pen upper body or housing 601 (such as an upper pen body or housing 1001, 2001, 3001, 4001, respectively), which houses a plurality of dose setting and injection components. The pen upper body 601 is connected to a cartridge housing 614, which houses a medication cartridge 615. The injection pen 600 may also include a lower pen cap 612 to cover the cartridge 615 and cartridge housing 614 when the injection pen is not in use. As shown, the injection pen 600 includes a dose set knob 602 (such as a dose set knob 1002, 2002, 3002, 4002, respectively) that includes a knob-like portion that is rotated by a user to set a desired dose. The dose set knob 602 also includes a plurality of indicators or numerals such as 1020, 2020, 3020, 4020, respectively, corresponding to a number of dosage units, that may be visible through a window 613 (such as window 1013, 2013, 3013, 4013, respectively) provided on the pen upper body 601 (such as an upper pen body or housing 1001, 2001, 3001, 4001, respectively), as shown in FIG. 20. A user rotates the dose set knob 602 (such as a dose set knob 1002, 2002, 3002, 4002, respectively) until the desired dose is visible in the window 613 (such as window 1013, 2013, 3013, 4013, respectively). The pen upper body 601 may include an arrow or other indicator 653 (such as indicator 1053, 2053, 3053, 4053, respectively) to precisely indicate the set dose. Once the desired dose is set, a user presses the button 603 (such as button 1003, 2003, 3003, 4003, respectively) until the set dosage amount is completely injected.

In an exemplary implementations, the interior of the push button 603 accommodates a clicker body 680 that engages an internal surface at a proximal end of a setback member or driver 609 and the dose set knob 602. The push button 603 is designed to rotate freely on a bushing insert on the clicker 680.

In an exemplary implementation, the setback member or driver 609 is a cylindrical member, as shown in FIG. 7, coaxial with and surrounded by the dose set knob 602. The setback member 609 is provided co-axially around a brake tower 605, as shown in FIG. 8, that is axially and rotatably fixed to the pen upper body 601. The brake tower 605 co-axially surrounds a non-rotatable hollow piston rod 606 and a tower core 620 disposed inside the piston rod 606, as shown in FIG. 53. The piston rod 606 includes a plurality of threads 662 provided on the interior surface thereof, as shown in FIG. 19. The piston rod 606 co-axially surrounds a lead screw 604 that includes a series of threads 642 at its distal end, as shown in FIG. 14. The lead screw threads 642 are in threaded engagement with the internal threads 662 provided on the piston rod 606. Due to its threaded engagement with the lead screw 604, the piston rod 606 is moved into the cartridge 615 during injection to press on a stopper 616 provided inside the cartridge 615 to expel a dose of medication. A wave spring or clip, or a foam ring, or such other biasing element 611, as shown in FIG. 7, can be positioned between a distal end of the brake tower 605 and the cartridge 615 to bias the cartridge 615 in a distal direction to prevent movement of the cartridge 615.

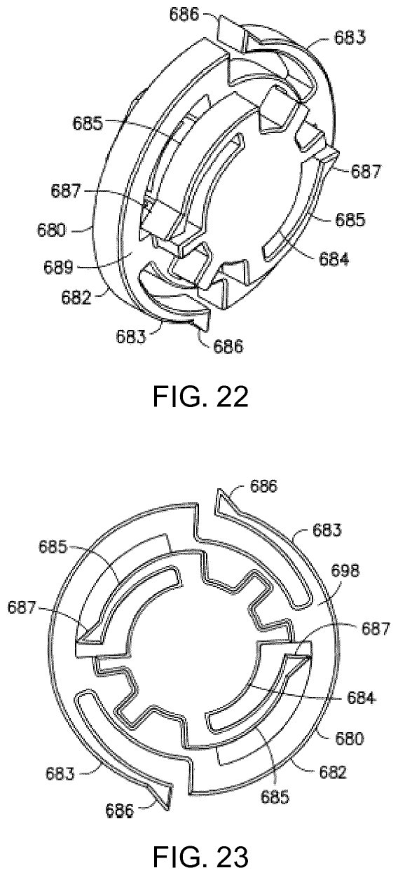

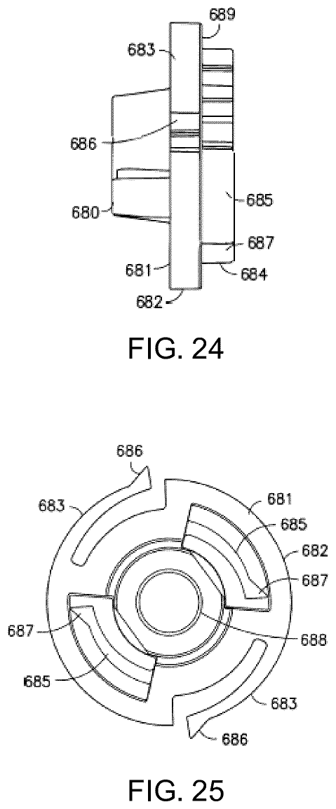

In an exemplary implementation, clicker body 680 is surrounded by the dose set knob 602, as shown in FIG. 8. An upper surface 681 of an upper ring 682 is engaged by a push button 603. A lower surface 689 of the upper ring 682 is engaged by a distal end 690 of a setback member 609. A pair of flexible arms 683 are connected to the upper ring 682, as shown in FIGS. 22, 23 and 25. A lower ring 684 is connected to the upper ring 682, as shown in FIG. 24. The lower ring 684 has a pair of flexible arms 685 connected thereto, as shown in FIGS. 22 and 23. Hooks 686 are disposed at free ends of the upper ring flexible arms 683, and hooks 687 are disposed at free ends of the lower ring flexible arms 687. An opening 688 is formed in the clicker body 680 to receive the push button 603. The upper ring flexible arm hooks 686 engage teeth 691 of the dose set knob 602. The lower ring flexible arm hooks 687 engage teeth 692 of the setback member 609.

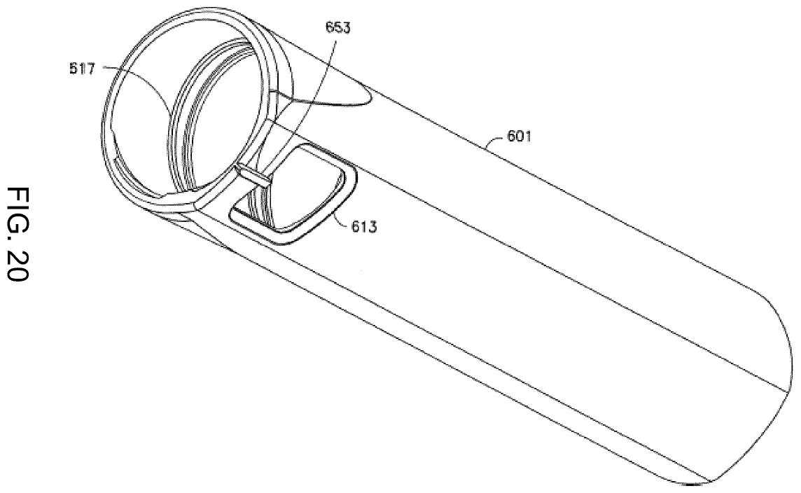

Tower core 620 is surrounded by piston rod 606, which is surrounded by the brake tower 605. Tower core 620 is provided axially and rotationally fixed to the brake tower 605. As shown in FIGS. 15 and 27-30, the tower core 620 has a key 623 extending axially at a proximal end. The key 623 is received by a V-shaped notch 653 disposed at a proximal end of the brake tower 605. The key 623 has inwardly tapering sides, as shown in FIGS. 27-29, to facilitate engagement with the V-shaped notch 653 of the brake tower 605, thereby rotationally locking the tower core 620 to the brake tower 605. The brake tower 605 is both axially and rotationally fixed to the pen upper body 601. As shown in FIG. 15, the tower core 620 is a substantially cylindrical element with an open side 624 extending along an axial length of the tower core 620. The open side 624 includes approximately one-fifth to one-quarter of the circumference of a cross section of the tower core 620. The open side 624 forms two longitudinally extending edges 625 and 626 at each end of the open side 624.

The tower core 620 functions to prevent rotation of the piston rod 606 relative to the brake tower 605 and thus the pen upper body 601. As shown in FIG. 8, the tower core 620 is surrounded by a hollow piston rod 606. The hollow piston rod 606 includes internal threads 662 extending along substantially an entire length of the hollow piston rod 606, as shown in FIGS. 18 and 19. The piston rod 606 is positioned with respect to the tower core 620 such that an internally extending key 661 engages the longitudinally extending edges 625 and 626, such that the piston rod 606 is prevented from rotating relative to the tower core 620, as shown in FIG. 30.

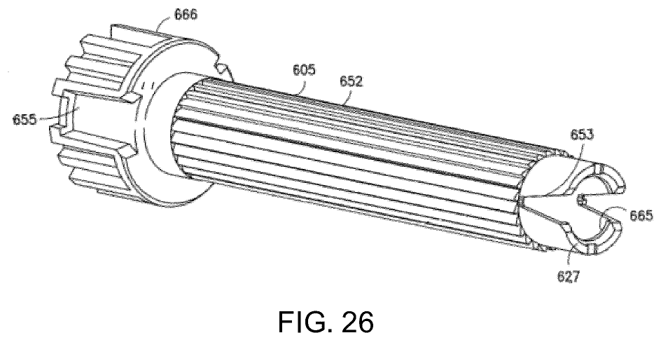

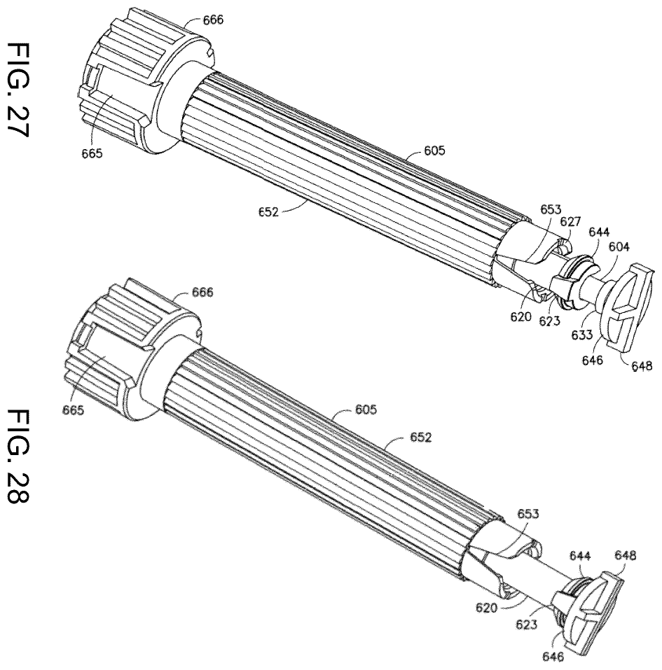

A lead screw 604 (FIG. 14) is provided in the interior of the hollow piston rod 606. A threaded portion 642 is provided at the distal end of the lead screw 604. The threaded portion 642 is configured to engage the internal threads 662 of the piston rod 606. The lead screw 604 is rotationally fixed to the setback member 609 such that rotation of the setback member 609 during an injection is transferred to the lead screw 604. The lead screw 604 is snapped into the tower core 620, which is snapped into the brake tower 605, as shown in FIGS. 8 and 27-30. A flange 633 of the lead screw 604 is received by a groove 632 (FIG. 15) of the tower core 620 such that a proximal end of the tower core 620 is received by an annular groove 645 of the lead screw 604 disposed between the proximal flange 646 and the flange 633 spaced inwardly therefrom. A flange 644 of the tower core 620 is received by an inwardly extending lip 665 (FIG. 26) of the brake tower 605. Axial movement of the lead screw 604 relative to the brake tower 605 is prevented in the distal direction by the flange 644 of the tower core 620 abutting the inwardly extending lip 665 of the brake tower 605. As such, due to the thread engagement between the threaded portion 642 of the lead screw 604 and the internal threads 662 on the hollow piston rod 606, relative rotation of the lead screw 604 with respect to the piston rod 606 (which is rotationally fixed to the tower core 620) drives the piston rod 606 axially in the distal direction inside the cartridge 615 to move the stopper 616 to expel medication contained therein.

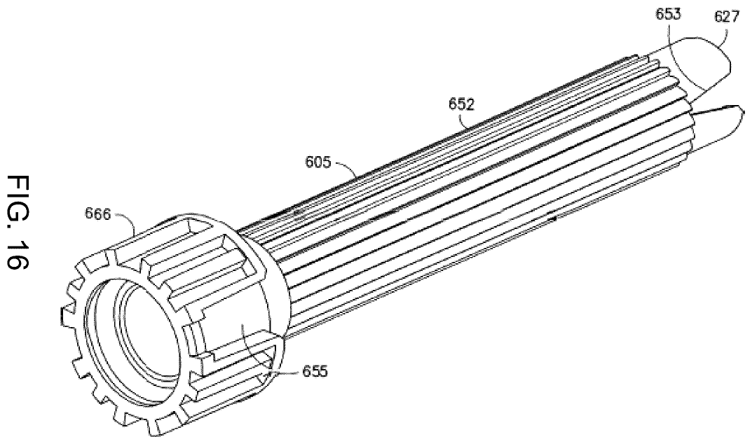

To set a dose using the injection pen 600, the user rotates the knob portion of the dose set knob 602 relative to the pen upper body 601. An outer surface 659 of the dose set knob 602 includes a thread 619, as shown in FIGS. 9 and 10, that is in threaded engagement with a plurality of threads 617 provided on the internal surface of the pen upper body 601, as shown in FIGS. 20 and 21. Accordingly, as the dose set knob 602 is rotated relative to the pen upper body 601, the dose set knob 602 screws or advances a distance out of the pen upper body 601. The dose set knob 602 includes an annular shoulder or rim 621 on the interior surface thereof near the proximal end. The annular shoulder 621 engages with an enlarged portion or head 699 (FIGS. 9-11) of the setback member 609, as shown in FIG. 8. The annular shoulder 621 of the dose set knob 602 may comprise a series of teeth or ridges 622 that engage with a plurality of similarly shaped teeth or ridges 698 provided on the enlarged head 699 of the setback member 609. The dose set knob teeth 622 and the setback member teeth 698 may extend in opposite axial directions. During dose setting, the dose set knob 602 is free to rotate with respect to the setback member 609 in both clockwise and counter-clockwise directions. As this occurs, the plurality of teeth or ridges 622 on the dose set knob 602 slip past the teeth 698 provided on the head portion 699 of the setback member 609, and in an exemplary implementation may thus provide a tactile signal or clicking noise to indicate the setting of a dosage amount. As further described below, the dose set knob 602 is enabled to rotate relative to the setback member 609 during dose setting due to a one-way ratchet that prevents the setback member 609 from rotating together with the dose set knob 602 in the setting direction.

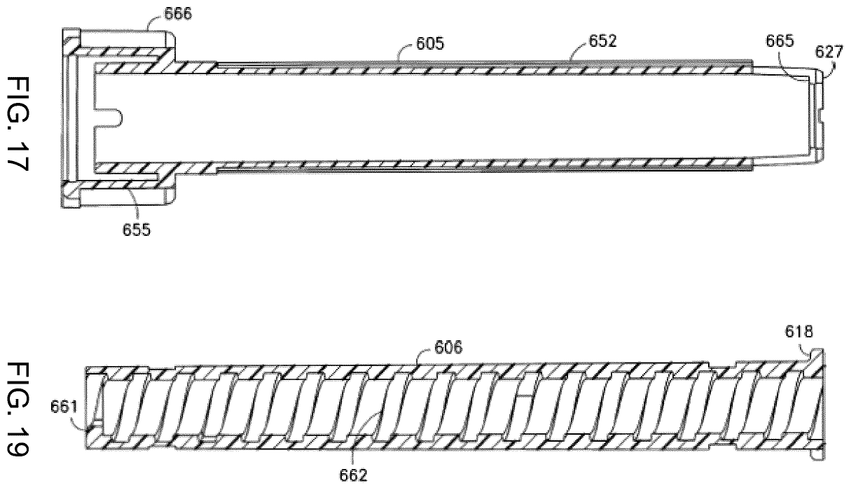

In an exemplary implementation, a clicker body 680 can be provided to facilitate generating a tactile signal or clicking noise during dose setting. The lower ring hooks 687 of the clicker body 680 are locked to the teeth 692 (FIGS. 11 and 12) of the setback member 609 such that the clicker body 680 is rotatably locked to the setback member 609. Rotation of the dose set knob 602 as the dose set knob 602 is advanced out of the pen upper body 601 to set the dose causes the teeth 691 (FIGS. 9 and 10) of the dose set knob 602 to slide over the upper ring hooks 686 of the clicker body 680, thereby generating a tactile signal and/or clicking noise to indicate to the user that a dose is being set.

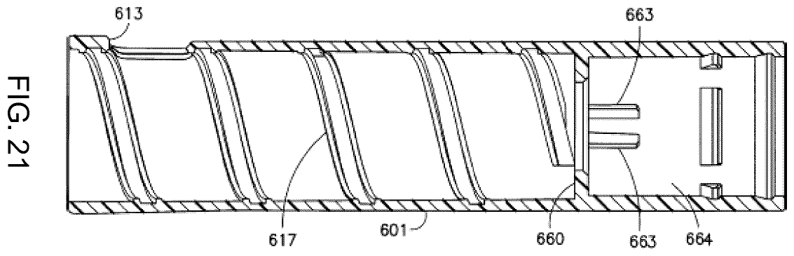

To correct a set dose that may have been set too high, the user simply rotates back the dose set knob 602 in the opposite direction. Rotation of the dose set knob 602 in this direction is not transferred to the setback member 609 due to the one-way ratchet between the setback member 609 and the brake tower 605. The setback member 609 has a pair of ratchet arms 696, as shown in FIGS. 11-13. The pair of ratchet arms 696 engages a plurality of splines or teeth 652 provided on the external surface of the brake tower 605, as shown in FIGS. 16 and 17. The ratchet arms 696 and splines or teeth 652 are configured to allow relative rotation in only one direction, namely, the direction that enables injection of a set dose. The friction provided between the ratchet arms 696 and the teeth 652 on the brake tower 605 is greater than the friction between the corresponding teeth 698 and 622 on the setback member 609 and the dose set knob 602, respectively. Thus, the dose set knob 609 can be rotated back to correct a set dose without causing rotation of the setback member 609 in this direction. Accordingly, the teeth 692 and 622 provided on the setback member 609 and dose set knob 602, respectively, slip past each other, and in an exemplary implementation may thus provide a tactile signal or clicking noise during dialing back of the dose, just as during normal dose setting, thereby indicating correction of the set dose.

In an exemplary implementation, the clicker body 680 can also facilitate generating a tactile signal or clicking noise during dose correcting. The upper ring hooks 686 of the clicker body 680 are locked to the teeth 691 of the dose set knob 602 such that the clicker body 680 is rotatably locked to the dose set knob. Rotation of the dose knob 602 back into the pen upper body 601 to correct the dose causes the teeth 692 of the setback member 609 to slide over the lower ring hooks 687 of the clicker body 680, thereby generating a tactile signal and/or clicking noise to indicate to a user that a dose is being corrected. Accordingly, the clicker body 680 facilitates generating a tactile signal and/or clicking noise during both dose setting and dose correcting.

As the dose set knob 602 screws or advances axially out of the upper body 601 during the setting of a dose, the setback member 609 is also caused to move axially out of the body by a corresponding distance. This axial movement is caused by the engagement between the annular shoulder 621 on the dose set knob 602 pushing against the enlarged head portion 699 of the setback member 609 during its movement out of the body.

Once a desired dose is set, the user pushes the push button 603 that is coupled to the clicker ring 680 that is axially connected to the setback member 609. Under the force applied by the user pressing the push button 603, the setback member 609 is moved into a locking or meshing engagement with the dose set knob 602 via a meshing of the respective teeth or ridges 698 and 622 provided on the dose set knob 602 and the setback member 609, respectively. As the user continues to press the push button 603, the dose set knob 602 is caused to rotate and screw back down into the pen upper body 601 via the thread engagement between the thread 619 on the dose set knob 602 and the thread 617 in the pen upper body 601. Rotation of the dose set knob 602 is then transferred to the setback member 609 due to their locking or meshed engagement. The force of the user pressing the button 603 is enough to overcome the friction between the ratchet arms 696 on the setback member 609 and the teeth or splines 652 on the brake tower 605. As a result, the setback member 609 is enabled to rotate in this direction. As the setback member 609 rotates relative to the brake tower 605 during injection, the ratchet arms 696 produce a tactile signal or clicking noise as they ratchet past the teeth 652 on the brake tower 605. This indicates to the user that injection of the set dose is taking place. Because the dose set knob 602 and the setback member 609 rotate together during the injection, the clicker body does not rotate relative to either the dose set knob 602 or the setback member 609. Accordingly, the clicker body 680 rotates with both the dose set knob 602 and the setback member 609 such that the clicker body 680 does not generate a tactile signal or clicking noise when injecting a set dose.

Rotation of the setback member 609, as allowed during injection, is then transferred to the lead screw 604, which is rotatably fixed to the setback member 609 via a key groove connection provided between the lead screw 604 and the setback member 609. As shown in FIGS. 11 and 12, an internal surface 668 of the setback member 609 includes a groove or slot 697 that is engaged with a key 648 provided at the proximal end of the lead screw 604, as shown in FIG. 14. The setback member 609 may include two oppositely disposed slots 697 for engaging two oppositely disposed keys 648 provided on the lead screw 604. The setback member 609 moves axially relative to the lead screw 604 during dose setting and dose correcting, via the key 648 and slot 697 interconnection. The length of the slot 697 in the setback member 609 may be configured to correspond to a maximum dose to be injected in a single injection. The lead screw 604 is axially fixed with respect to the pen upper body 601 via a snap engagement described above with the brake tower 605, which is axially and rotatably fixed to the pen upper body 601. As shown in FIGS. 14 and 15, the lead screw 604 includes the inwardly disposed flange 633 that is received by the recess 632 in the tower core 620. The flange 644 of the tower core 620 is received by the inwardly extending lip 665 of the brake tower 605 (FIG. 26), thereby axially locking the lead screw 604 to the brake tower 605 and the pen upper body 601.

The lead screw 604 includes a plurality of threads 642 at its distal end that are in threaded engagement with the internal threads 662 which may be provided along the entire length of the hollow piston rod 606, as shown in FIGS. 8 and 19. The piston rod 606 is held non-rotatable with respect to the pen upper body 601 due to the engagement between the piston rod key 661 and the outer edges 625 and 626 of the tower core 620, as shown in FIG. 30. The piston rod key 661 is guided in its axial movement by the axially extending outer edges 625 and 626 of the tower core 620, thereby preventing relative rotation therebetween while permitting the piston rod 606 to move axially with respect thereto. As the setback member 609 does not rotate during dose setting and correcting, the lead screw 604 does not rotate during dose setting and correcting, which prevents movement of the piston rod 606 during dose setting and correcting. Accordingly, rotation of the lead screw 604 during injection of a dose causes the threads 642 of the lead screw 604 to engage the threads 662 of the piston rod 606, thereby axially moving the piston rod 606.

In an exemplary implementation, during assembly, the brake tower 605 is inserted into the pen upper body 601 from the distal end. As shown in FIGS. 8 and 21, the pen upper body 601 includes a transverse wall 660 that limits the movement of the brake tower 605 into the body 601 by blocking an enlarged distal portion 666 of the brake tower 605. Further, an inwardly protruding key 663 is also provided distally from the transverse wall 660 on an internal surface 664 of the pen upper body 601, as shown in FIG. 21. The key 663 engages with a slot 655 provided on the enlarged distal portion 666 of the brake tower 605, as shown in FIGS. 16 and 17, to rotationally fix the brake tower 605 with respect to the pen upper body 601. A plurality of axially extending keys 663 may be disposed on the inner surface of the pen upper body 601 to engage a plurality of slots 655 on the enlarged distal portion 666 of the brake tower 605.

Because the piston rod 606 is non-rotatable with respect to the body 601, as the lead screw 604 is caused to rotate during injection, as described above due to its rotational coupling with setback member 609, the piston rod 606 through its threaded engagement with lead screw 604 is caused to move in the distal direction such that a piston rod flange 618 presses against the stopper 616 provided in the medicament cartridge 615, thus expelling a liquid medication therefrom. The piston rod 606 is prevented from moving in the proximal direction because the lead screw 604 is rotatable in only a single direction (that which results in distal movement of the piston rod 606) due to the one-way ratchet between the setback member 609 and the brake tower 605.

In an exemplary implementation, a dose stop member 607, as shown in FIG. 8, is provided for last dose management, to prevent the setting of a dose that is larger than the remaining amount of medication in the cartridge 615. The dose stop member 607 is axially slidable but rotationally fixed with respect to the setback member 609 by being positioned between a pair of splines 694 provided on the outer surface of the setback member 609. The dose stop member 607 is a half-nut like element that is threaded on its outer surface with a plurality of threads 672. These threads 672 are configured to engage with corresponding threads 674 provided on the interior of the dose set knob 602, as shown in FIG. 10. During dose setting, as the dose set knob 602 rotates relative to the setback member 609, and therefore also relative to the dose stop member 607, the dose stop member 607 is caused to slide in the distal direction by a distance corresponding to the set dose due to its engagement with the threads 674 in the dose set knob 602.

During injection, because the setback member 609 and the dose set knob 602 are rotationally coupled as discussed above, the dose stop member 607 will maintain its position relative to the threads 674 of the dose set knob 602. The dose stop member 607 will move in the distal direction during dose setting until a distal edge 673 of the dose stop member 607 abuts an inwardly directed key 675 provided on the internal surface of the dose set knob 602, as shown in FIG. 10. In this position, the dose stop member 607 is prevented from further movement in the distal direction which also prevents further rotation of the dose set knob 602 to set an additional dose.

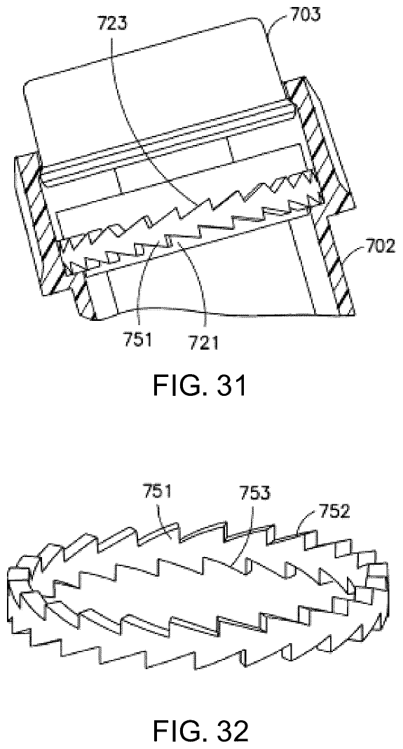

FIGS. 31-35 illustrate another example embodiment of an injection pen with similar functionality to the injection pen described with respect to FIGS. 7-21 and 26-30. The example embodiment depicted in FIGS. 31-35 includes a modified clicker body 751 that replaces the clicker body 780 of FIGS. 7-21 and 26-30. The remaining components and functions of the injection pen are substantially similar to the injection pen 600.

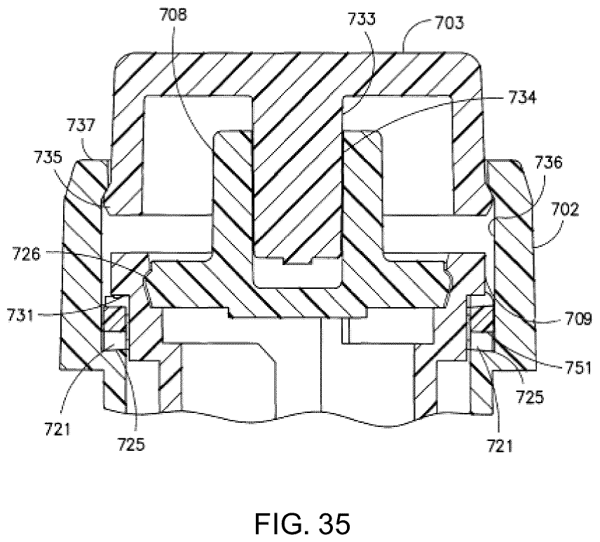

The clicker body 751 is substantially ring-shaped having an upper set of teeth 752 and a lower set of teeth 753, as shown in FIGS. 31 and 32. The upper teeth 752 may have a slope that is opposite that of the lower teeth 753. The sloped surfaces of the upper teeth 752 and the lower teeth 753 may form an angle of approximately 15 degrees. As shown in FIGS. 31 and 35, the clicker body 751 is disposed between an annular shoulder 725 of the dose set knob 702 and an enlarged portion 731 of the setback member 709. A plurality of teeth 721 extend axially in the proximal direction from the shoulder 725 of the dose set knob 702. A plurality of teeth 723 extend axially in the distal direction from the enlarged portion 731 of the setback member 709. A bearing insert 708 is received in an annular groove 726 of the setback member 709, as shown in FIG. 35. A push button 703 (such as 1003, 2003, 3003, 4003) has a projection 733 received by an opening 734 in the bearing insert 708. A distal skirt 735 of the push button 703 is slidably received by a recess 736 adjacent a proximal end 737 of the dose set knob 702.

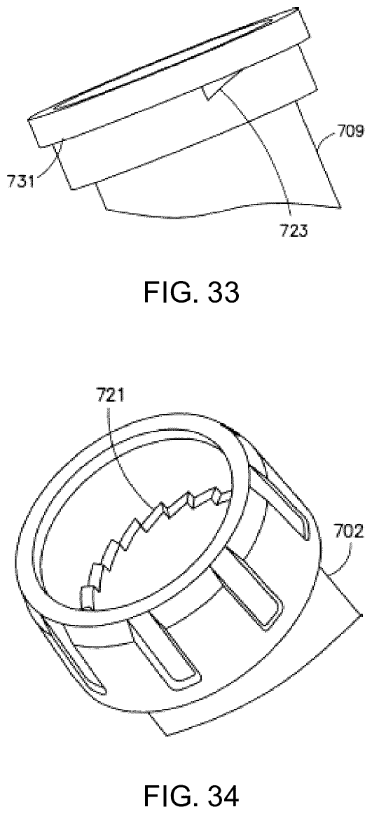

The clicker body 751 facilitates generating a tactile signal or clicking noise during dose setting. The upper teeth 752 of the clicker body 751 are locked to the teeth 721 (FIG. 34) of the dose set knob 702 such that the clicker body 751 rotates with the dose set knob 702 as the dose set knob 702 advances out of the pen upper body. The lower teeth 753 slide over the teeth 723 (FIG. 33) of the setback member 709. Accordingly, a tactile signal or clicking noise is generated to indicate to the user that a dose is being set.

The clicker body 751 also facilitates generating a tactile signal or clicking noise during dose correcting. The lower teeth 753 of the clicker body 751 are locked to the teeth 723 (FIG. 33) of the setback member 709 such that the clicker body 751 is rotatably locked to the setback member 709. Rotation of the dose set knob 702 as the dose set knob 702 is advanced back into pen upper body to correct the dose causes the teeth 721 (FIG. 34) of the dose set knob 702 to slide over the lower teeth 753 of the clicker body 751, thereby generating a tactile signal or clicking noise to indicate to the user that a dose is being corrected. Accordingly, the clicker body 751 facilitates generating a tactile signal or clicking noise during both dose setting and dose correcting.

Because the dose set knob 702 and the setback member 709 rotate together during an injection, the clicker body 751 does not rotate relative to either the dose set knob 702 or the setback member 709. Accordingly, the clicker body 751 rotates with both the dose set knob 702 and the setback member 709 such that the clicker body 751 does not generate a tactile signal or clicking noise when injecting a set dose.

It may be understood that the example embodiments described herein may be considered in a descriptive sense only and not for purposes of limitation. Descriptions of features or aspects within each example embodiment may be considered as available for other similar features or aspects in other example embodiments.

While example embodiments have been described with reference to the figures, it will be understood by those of ordinary skill in the art that various changes in form and details may be made therein without departing from the spirit and scope as defined by the following claims.

1. A medication injection method comprising:
moving a dose set knob of a medication injection pen with respect to a housing of the medication injection pen to set a desired dose, the desired dose comprising a value and the dose set knob comprising at least one indicator of said value of the desired dose; and
positioning said at least one indicator of said value with respect to an opening in the housing by said moving,
wherein said indicator is legible as said value of the desired dose only when viewed through said opening, and said indicator being legible signifies that the desired dose is set.

2. The medication injection method according to claim 1, wherein the opening comprises a window and a cover disposed in the window, the method further comprising displaying said indicator of said value through cover, whereby said representation is legible as said value of the desired dose when viewed through said cover.

3. The medication injection method according to claim 2, wherein the cover comprises an opaque cover having seven transparent portions for each numeric digit of said value of said desired dose, and said at least one indicator comprises markings disposed on said dose set knob indicative of said value of said desired dose when viewed through said cover.

4. The medication injection method according to claim 2, wherein the cover comprises a lens, and said at least one indicator is dispose on said dose set knob and not legible when viewed outside said lens.

5. The medication injection method according to claim 4, wherein said lens is a convex lens and said indicator comprises a small-font printed value less legible when viewed outside said lens.

6. The medication injection method according to claim 2, wherein the cover comprises a polarized filter, and said at least one indicator comprises a numeric representation of said value of said desired dose disposed on said dose set knob, said representation being legible as said value of said desired dose when viewed through said cover.

7. The medication injection method according to claim 2, wherein the cover comprises a color filter, and said at least one indicator comprises a numeric representation of said value of said desired dose disposed on said dose set knob within a colored field, said representation being legible as said value of said desired dose when viewed through said cover.

8. The medication injection method according to claim 1, wherein said moving comprises axially displacing the dose set knob with respect to the housing.

9. The medication injection method according to claim 8, wherein said moving further comprises rotating said dose set knob with respect to said housing to cause said axial displacement.

10. The medication injection method according to claim 1, further comprising injecting the set desired dose.

11. A medication injection pen comprising:
a housing having an opening therein; and
a dose set knob, disposed at least partially within the housing and movable with respect to the housing to set a desired dose, the desired dose comprising a value and the dose set knob comprising at least one indicator of said value of the desired dose;
wherein said indicator is legible as said value of the desired dose only when viewed through said opening, and said indicator being legible signifies that the desired dose is set.

12. The medication injection pen according to claim 11, wherein the opening comprises a window and a cover disposed in the window, whereby said representation is legible as said value of the desired dose when viewed through said cover.

13. The medication injection pen according to claim 12, wherein the cover comprises an opaque cover having seven transparent portions for each numeric digit of said value of said desired dose, and said at least one indicator comprises markings disposed on said dose set knob indicative of said value of said desired dose when viewed through said cover.

14. The medication injection pen according to claim 12, wherein the cover comprises a lens, and said at least one indicator is dispose on said dose set knob and not legible when viewed outside said lens.

15. The medication injection pen according to claim 14, wherein said lens is a convex lens and said indicator comprises a small-font printed value less legible when viewed outside said lens.

16. The medication injection pen according to claim 12, wherein the cover comprises a polarized filer, and said at least one indicator comprises a numeric representation of said value of said desired dose disposed on said dose set knob, said representation being legible as said value of said desired dose when viewed through said cover.

17. The medication injection pen according to claim 12, wherein the cover comprises a color filter, and said at least one indicator comprises a numeric representation of said value of said desired dose disposed on said dose set knob within a colored field, said representation being legible as said value of said desired dose when viewed through said cover.

18. The medication injection pen according to claim 11, wherein the dose set knob is axially displaceable with respect to the housing.

19. The medication injection pen according to claim 18, wherein the dose set knob is rotatable with respect to said housing, and rotating the dose set knob with respect to said housing causes said axial displacement.

20. The medication injection pen according to claim 12, further comprising means for at least one of setting the desired dose, correcting to the desired dose, and injecting the desired dose.
